(19) United States
(12) Patent Application Publication
(10) Pub. No.: US 2012/0094931 A1
(43) Pub. Date: Apr. 19, 2012

(54) COMPOSITIONS AND METHODS TO MODULATE PROGRESSION AND ONSET OF INFLAMMATORY BOWEL DISEASE

(76) Inventors: Colm COLLINS, Denver, CO (US); Jesus RIVERA-NIEVES, Denver, CO (US); Charles A. DINARELLO, Boulder, CO (US)

(21) Appl. No.: 13/171,353

(22) Filed: Jun. 28, 2011

Related U.S. Application Data
(60) Provisional application No. 61/360,409, filed on Jun. 30, 2010.

(51) Int. Cl. A61K 38/57; A61P 29/00; A61P 1/00

(52) U.S. Cl. 514/20.3

(57) ABSTRACT

Embodiments herein illustrate methods and compositions for treating inflammatory bowel disorders. In certain embodiments, compositions and methods relate to reducing, inhibiting or modulating progression of an inflammatory bowel disorder in a subject. Other embodiments herein relate to compounds including naturally occurring and synthetic mutant compositions of alpha-1 antitrypsin.

### CROSS REFERENCE TO RELATED APPLICATION

This application claims the benefit under 35 U.S.C. §119(e) of provisional application Ser. No. 61/360,409 filed Jun. 30, 2010, this application is incorporated herein by reference in its entirety for all purposes.

### FIELD

Embodiments herein relate to compositions, methods and uses for alpha-1 antitrypsin (α-1 antitrypsin, AAT) or derivative or analog thereof for the treatment or prevention of bowel disease. In certain embodiments, treatment or prevention of bowel disease includes, but is not limited to, treatment or prevention of inflammatory bowel disease (IBS or IBD). In other embodiments, IBS can be ulcerative colitis and/or Crohn's disease (CD). Some embodiments report AAT or peptide derivative thereof may be used to treat or prevent IBS or IBD in a subject. Yet other embodiments herein concern reducing side effects of these conditions in a subject.

### BACKGROUND

Normal plasma concentration of alpha-1 antitrypsin (AAT) ranges from 1.3 to 3.5 mg/ml. Under certain conditions, AAT can behave as an acute phase reactant and increase 3-4-fold during host response to inflammation and/or tissue injury or dramatic change such as with pregnancy, acute infection, and tumors. AAT easily diffuses into tissue spaces and forms a 1:1 complex with target proteases, principally neutrophil elastase. Other enzymes such as trypsin, chymotrypsin, cathepsin G, plasmin, thrombin, tissue kallikrein, and factor Xa can also serve as substrates. The enzyme/inhibitor complex is then removed from circulation by binding to serpin-enzyme complex (SEC) receptor and catabolized by the liver and spleen.

Crohn's disease (CD) and ulcerative colitis (UC) are inflammatory bowel diseases. UC is further characterized by superficial fissuring ulceration of the colonic mucosa with little evidence of inflammatory infiltration of the underlying muscularis. Ileal and colonic inflammation have been observed in these disorders, as well as acute and chronic intestinal immune responses.

### SUMMARY

Embodiments herein provide for methods and compositions for treating a subject having a bowel disorder. In certain embodiments, compositions and methods herein report modulating inflammatory bowel disorder. Some embodiments herein report modulating one or more symptom of a bowel disorder. In accordance with these embodiments, symptoms of a bowel disorder may be treated in a subject having the bowel disorder to modulate weight loss, modulate disease activity (e.g. measured by a disease activity index), modulate colon shortening, modulate visible observations of the disorder (e.g. changes in intestinal mucosa) or a combination thereof. Some embodiments of the present invention concern modulating the onset or progression of an inflammatory bowel disorder.

Certain embodiments concern compositions for treating a subject having an inflammatory bowel disorder. In accordance with these embodiments, a composition can include, alpha-1 antitrypsin, or fragments, or derivatives thereof, or mutants thereof. Some embodiments concern administering naturally occurring AAT to a subject having or expected to develop an inflammatory bowel disorder.

Compositions contemplated herein may further include an agent selected from the group consisting of an anti-inflammatory agent, an immunosuppressive agent, an immunomodulatory agent, an anti-viral agent, an anti-pathogenic agent, an anti-bacterial agent, a protease inhibitor, and a combination thereof.

In certain embodiments, compositions herein can be administered orally, systemically, via an implant, time released or slow-release compositions (e.g. gel, microparticles etc.), intravenously, topically, intrathecally, subcutaneously, by inhalation, nasally, or by other means known in the art or a combination thereof.

Some embodiments herein concern compositions of use to treat an inflammatory bowel disorder where the composition to treat the condition has reduced or eliminated serine protease activity.

In certain embodiments, compositions and methods disclosed herein can be used to reduce or prevent onset of inflammatory bowel disorder in a subject. In accordance with these embodiments, reduction in conditions associated with IBS in a subject may be on the order of about 10-20%, or about 30-40%, or about 50-60%, or about 75-100% reduction or inhibition. In accordance with these embodiments, a subject having IBS or IBD may be treated with a pharmaceutically acceptable composition of AAT or AAT-carboxyterminal peptide to reduce wasting or to reduce loss of or restore barrier function compared to a control subject not receiving such a composition.

Some embodiments herein concern restoring bowel or intestinal hyperpermeability in a subject having an acute or chronic condition. In accordance with these embodiments bowel or intestinal hyperpermeability or loss of barrier function can be due to chronic diseases such as systemic inflammatory response syndrome (SIRS), inflammatory bowel disease, type 1 diabetes, allergies, and asthma. In certain embodiments, a subject having bowel or intestinal hyperpermeability can be treated by a health professional by a predetermined regimen such as daily, twice weekly, weekly or other predetermined regimen.

In certain embodiments, α1-antitrypsin used in the methods and compositions herein can include, but is not limited to, naturally occurring AAT (394 AA, makes up about 90% of AAT derived from human platelets), Aralast™ (Baxter), Zemaira™ (Aventis Behring), Prolastin™ and Prolastin C™ (Talecris, N.C.), Aprotonin™ or Trasylol™ (Bayer Pharmaceutical Corporation) and Ulinistatin™ (Ono Pharmaceuticals, Inc.), Kamada AAT (Kamada, Inc., Israel) or any combination thereof. In other embodiments, AAT or an AAT fragment or an AAT analog used in methods and compositions herein can include naturally occurring AAT or AAT fragment or analog or allele thereof.

In other embodiments, compositions herein may include an anti-inflammatory compound or immunomodulatory agent. In accordance with these embodiments, these agents can include, but are not limited to, interferon; interferon derivatives comprising betaseron, β-interferon; prostane derivatives comprising iloprost, cicaprost; glucocorticoids comprising cortisol, prednisolone, methylprednisolone, dexamethasone; immunosuppressive comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukines, other cytokines, T-cell-proteins; antibodies against receptors of interleukines, other cytokines, T-cell-proteins; and calcipotriols and analogues thereof taken either alone or in any combination thereof.

In certain embodiments, compositions for administration can be in a range of between about 10 ng and about 10 mg per ml or mg of the formulation. In certain methods, a regimen can include administering a composition of about 1 mg/kg to about 150 mg/kg or about 10 mg/kg to about 100 mg/kg daily, weekly, monthly or as determined by a health professional evaluating the needs of a subject having a bowel disorder or other condition. A therapeutically effective amount of AAT peptides or drugs that have similar activities as AAT or peptides drug may be measured in molar concentrations and may range between about 1 nM and about 10 mM. The formulation is also contemplated in combination with a pharmaceutically or cosmetically acceptable carrier. Precise doses can be established by well known routine clinical trials without undue experimentation.

In some embodiments, pharmaceutical compositions contemplated herein are administered orally, systemically, via an implant, intravenously, topically, intrathecally, intracranially, intraventricularly, by inhalation or nasally.

In certain embodiments, the subject or mammal is a human.

In other embodiments, the subject or mammal can be a domesticated or a non-domesticated mammal.

In certain embodiments, synthetic and/or naturally occurring peptides may be used in compositions and methods herein for example, providing other than serine protease inhibitor activity of AAT. Homologues, natural peptides derivatives, with sequence homologies to AAT including peptides directly derived from cleavage of AAT may be used or other peptides such as, peptides that have AAT-like activity other than serine protease inhibitor activity. Other peptidyl derivatives, e.g., aldehyde or ketone derivatives of such peptides are also contemplated herein. Without limiting to AAT and peptide derivatives of AAT, compounds like oxadiazole, thiadiazole and triazole peptoids and substances can include, but are not limited to, certain phenylenedialkanoate esters, CE-2072, UT-77, and triazole peptoids. Examples of analogues are TLCK (tosyl-L-lysine chloromethyl ketone) or TPCK (tosyl-L-phenylalanine chloromethyl ketone) or any combination thereof.

In certain embodiments, compositions comprising human AAT mutants can be generated having no significant serine protease inhibitor activity of use in methods described herein. In other embodiments, constructs of human AAT mutants having no significant serine protease activity can be associated with a vector. Vectors include, but are not limited to, genetic therapy vectors (e.g. EF vector of Epstein Barr Virus). Other embodiments concern AAT-derived fragment constructs adapted to have no significant serine protease inhibitor activity. In accordance with these embodiments, constructs can be generated and amplified to produce large quantities of AAT having no significant serine protease inhibitor activity. Industrial sized scale production is contemplated in order to produce large quantities of AAT having no significant serine protease inhibitor activity for use in treatment and procedures contemplated herein. In certain embodiments, attenuated viruses may be used to produce an AAT construct. In other embodiments, attenuated viruses or viral fragments known in the art may be used to introduce AAT or AAT derivatives or peptide constructs having no significant serine protease activity to a subject using, for example, gene therapy techniques.

In certain embodiments of the present invention, an anti-inflammatory agents or immunomodulatory agents can be included in any of the compositions disclosed. These agents include, but are not limited to, one or more of interferon, interferon derivatives including betaseron, beta-interferon, prostane derivatives including iloprost, cicaprost; glucocorticoids including cortisol, prednisolone, methyl-prednisolone, dexamethasone; immunsuppressives including cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives including ACTH and analogs thereof; soluble TNF-receptors; TNF-antibodies; soluble receptors of interleukins, other cytokines, T-cell-proteins; antibodies against receptors of interleukins, other cytokines, T-cell-proteins; and calcipotriols; Celcept®, mycophenolate mofetil, and analogues thereof taken either alone or in combination.

In certain embodiments, synthetic and/or naturally occurring peptides may be used in compositions and methods disclosed in embodiments herein. Homologues, natural peptides, derived from AAT including peptides directly derived from cleavage of AAT may be used or other peptides such as, peptides that inhibit serine proteases or have AAT-like activity. Other peptidyl derivatives, e.g., aldehyde or ketone derivatives of such peptides are also contemplated herein. Without limiting to AAT and peptide derivatives of AAT, compounds like oxadiazole, thiadiazole and triazole peptoids and substances comprising certain phenylenedialkanoate esters, CE-2072, UT-77, and triazole peptoids may be used. Examples of analogues are TLCK (tosyl-L-lysine chloromethyl ketone) or TPCK (tosyl-L-phenylalanine chloromethyl ketone).

In one aspect of the invention, pharmaceutical compositions can be administered orally, systemically, via an implant, intravenously, topically, intrathecally, intratracheally, intracranially, subcutaneously, intravaginally, intraventricularly, intranasally such as inhalation, or any combination thereof.

Other embodiments concern methods for preventing or reducing onset or progression of a bowel disorder, the method including administering to the subject a composition having AAT, a mutant AAT construct, and a pharmaceutically acceptable carrier to a subject. Any composition disclosed herein can be administered to the subject before, during, and/or after a subject having a bowel disorder in the subject. A composition contemplated herein can further include one or more anti-inflammatory agent, immunosuppressive agent, immunomodulatory agent, anti-microbial agent, or a combination thereof.

Immunosuppressive agent can be chosen from inhibitors of apoptosis, reducers of lymphocyte numbers, reducers of cytokine production, reducers of cytokine activities, monoclonal antibodies, reducers of cytokine receptors, reducers of nitric oxide production and a combination thereof.

Other agents contemplated herein can include reducers of cytokine production, reducers of cytokine activities, reducers of cytokine receptors is an inhibitor of one or more of TNFα (tumor necrosis factor alpha), IL-1 (interleukin-1), IL-12 (interleukin-12), IL-18 (interleukin-18), IL-17 (interleukin-17), IL-23 (interleukin-23), IL-32 (interleukin-32), IFNγ (interferon gamma) or a combination thereof.

Other embodiments herein include treating bowel disorder in a subject by identifying a subject having or developing a bowel disorder; administering a therapeutically effective amount of a composition comprising AAT, AAT derivative having no significant serine protease inhibitor activity, AAT-like compound, AAT analog, AAT derivative, or combination thereof to the subject. Administering the composition may include administering the composition directly to the region affected by disorders or other delivery methods such as intravenously.

In certain embodiments, the subject is a human. In some embodiments, the subject is a domesticated animal or livestock.

As such, those skilled in the art will appreciate that the conception, upon which this disclosure is based, can readily be used as a basis for designing other methods for carrying out the several features and advantages of embodiments of the present invention.

### BRIEF DESCRIPTION OF THE DRAWINGS

The following drawings form part of the present specification and are included to further demonstrate certain embodiments disclosed herein. Embodiments may be better understood by reference to one or more of these drawings in combination with the detailed description of specific embodiments presented herein.

FIGS. 1A-1D represents a mouse model of acute colitis where A and B represent exemplary plots of effects of various treatments on weight loss over time; C represents an exemplary histogram plot of colon length changes using various treatments and D represents a macroscopic view of experimental colons after treatment.

FIGS. 2A-2D represent histogram plots related to cytokine release in colon explants of treated and untreated mice.

FIGS. 3A-3C represent levels of T lymphocytes in various internal locations of treated and untreated mice.

FIGS. 4A-4F represent plots and histograms of recovery of a subject post insult either untreated or treated with various compositions disclosed herein.

FIGS. 5A and 5B represent a comparative analysis of lymphocyte profiles in a mouse model post insult (IBD mouse model).

FIGS. 6A and 6B represent a comparative analysis of lymphocyte profiles in a mouse model post insult (IBD mouse model).

FIGS. 7A and 7B represent a comparison of a model of attenuated inflammation with related subjects.

FIG. 8 represents a mouse model comparison of littermates using control and experimental parameters regarding levels of T lymphocytes in various locations in vivo.

FIG. 9 represents the effects of various compositions disclosed herein on intestinal permeability at 0 and 8 days.

FIG. 10 represents a section of intestinal lining with and without treatment with compositions disclosed herein.

### DESCRIPTION OF ILLUSTRATIVE EMBODIMENTS

### Definitions

As used herein, “a” or “an” may mean one or more than one of an item.

As used herein, “about” can mean plus or minus 10%, for example, about 10 minutes can mean from 9 to 11 minutes.

### DETAILED DESCRIPTION

In the following sections, various exemplary compositions and methods are described in order to detail various embodiments of the invention. It will be obvious to one skilled in the art that practicing the various embodiments does not require the employment of all or even some of the specific details outlined herein, but rather that concentrations, times and other specific details may be modified through routine experimentation. In some cases, well known methods, or components have not been included in the description.

Some of the main forms of IBD are Crohn's disease and ulcerative colitis (UC). Accounting for far fewer cases are other forms of IBD: collagenous colitis, lymphocytic colitis, ischaemic colitis, diversion colitis, behçet's syndrome and indeterminate colitis.

Some of the main differences between Crohn's disease and UC are the location and nature of the inflammatory changes. Crohn's can affect any part of the gastrointestinal tract, from mouth to anus (skip lesions), although a majority of the cases start in the terminal ileum. Ulcerative colitis, in contrast, is restricted to the colon and the rectum. Microscopically, ulcerative colitis is restricted to the mucosa (epithelial lining of the gut), while Crohn's disease affects the whole bowel wall. Crohn's disease and ulcerative colitis can present with extra-intestinal manifestations (such as liver problems, arthritis, skin manifestations and eye problems) in different proportions. It is rare that a definitive diagnosis of neither Crohn's disease nor ulcerative colitis can be made because of idiosyncrasies in the presentation. In this case, a diagnosis of indeterminate colitis may be made. Although a recognised definition, not all centres refer to this.

Microscopically, ulcerative colitis is restricted to the mucosa (epithelial lining of the gut), while Crohn's disease affects the whole bowel wall. Crohn's disease and ulcerative colitis present with extra-intestinal manifestations (e.g. liver problems, arthritis, skin manifestations and eye problems) in different proportions.

Rarely, a definitive diagnosis of neither Crohn's disease nor ulcerative colitis can be made because of idiosyncrasies in the presentation. In this case, a diagnosis of indeterminate colitis may be made. Although a recognised definition, not all centres refer to this.

Inflammatory bowel disease is characterized by superficial fissuring ulceration of the colonic mucosa with little evidence of inflammatory infiltration of the underlying muscularis. There is initial flaring insult but also the intestinal response that drives the mucosal healing process.

Some side effects of intestinal and other chronic disorders can include leaky gut. This term is a name used to describe intestinal or bowel hyperpermeability. Tight junctions represent one of the major barriers within the pathway between intestinal epithelial cells that line the digestion tract. Disruption of tight junctions can lead to intestinal hyperpermeability that has some relationship with acute and chronic diseases such as systemic inflammatory response syndrome (SIRS), inflammatory bowel disease, type 1 diabetes, allergies, and asthma. A lack of mucosal integrity with consecutive local and systemic inflammation and dysfunction of transport proteins may worsen the clinical symptoms of chronic heart failure. A ‘leaky’ bowel wall may lead to translocation of bacteria and/or endotoxin, which may be an important stimulus for inflammatory cytokine activation. Other side effects of an intestinal disorder can include weight loss and wasting of a subject having such a condition.

Embodiments herein provide for methods and compositions for treating a subject having a medical disorder. In other embodiments, medical disorders can include a chronic or acute intestinal disorder. In accordance with these embodiments, the composition may include, alpha-1 antitrypsin, an analog thereof, or fusion molecule thereof. In other embodiments, a composition may further include, but is not limited to, an anti-inflammatory agent, an immunosuppressive agent, an immunomodulatory agent, an anti-microbial agent, an anti-viral agent, an anti-bacterial agent, and a combination thereof.

Any of the embodiments detailed herein may further include one or more a therapeutically effective amount of anti-microbial drugs anti-inflammatory agent, immunomodulatory agent, or immunosuppressive agent or combination thereof.

In addition, other combination compositions of methods disclosed in the present invention include certain antibody-based therapies. Non-limiting examples include, polyclonal anti-lymphocyte antibodies, monoclonal antibodies directed at the T-cell antigen receptor complex (OKT3, TIOB9), monoclonal antibodies directed at additional cell surface antigens, including interleukin-2 receptor alpha.

In one embodiment, the reduction, prevention or inhibition of onset or progression of a bowel disorder condition may be about 10-20%, 30-40%, 50-60%, or more due to administration of disclosed compositions herein.

### Pharmaceutical Compositions

Embodiments herein provide for administration of compositions to subjects in a biologically compatible form suitable for pharmaceutical administration in vivo. By “biologically compatible form suitable for administration in vivo” is meant a form of the active agent (e.g. pharmaceutical chemical, protein, gene, antibody etc of the embodiments) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically active amount of a compound may vary according to factors such as the disease state, age, sex, and weight of the individual, and the ability of antibody to elicit a desired response in the individual. Dosage regima may be adjusted to provide the optimum therapeutic response.

In one embodiment, the compound (e.g. pharmaceutical chemical, protein, peptide etc. of the embodiments) may be administered in a convenient manner such as subcutaneous, intravenous, by oral administration, inhalation, transdermal application, intravaginal application, topical application, intranasal or rectal administration. Depending on the route of administration, the active compound may be coated in a material to protect the compound from the degradation by enzymes, acids and other natural conditions that may inactivate the compound. In a preferred embodiment, the compound may be orally administered. In another preferred embodiment, the compound may be administered intravenously. In one particular embodiment, the compound may be administered intranasally, such as by inhalation to a subject.

A compound may be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. It may be necessary to coat the compound with, or co-administer the compound with, a material to prevent its inactivation. The active agent may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use may be administered by means known in the art. For example, sterile aqueous solutions (where water soluble) or dispersions and sterile powders for the extemporaneous preparation of sterile injectable solutions or dispersion may be used. In all cases, the composition can be sterile and can be fluid to the extent that easy syringability exists. It might be stable under the conditions of manufacture and storage and may be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of microorganisms can be achieved by heating, exposing the agent to detergent, irradiation or adding various antibacterial or antifungal agents.

Sterile injectable solutions can be prepared by incorporating active compound (e.g. a compound that reduces serine protease activity) in the required amount in an appropriate solvent with one or a combination of ingredients enumerated above, as required, followed by filtered sterilization.

Aqueous compositions can include an effective amount of a therapeutic compound, peptide, epitopic core region, stimulator, inhibitor, and the like, dissolved or dispersed in a pharmaceutically acceptable carrier or aqueous medium. Compounds and biological materials disclosed herein can be purified by means known in the art.

Solutions of the active compounds as free-base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. Prolonged absorption of the injectable compositions can be brought about by the use in the compositions of agents delaying absorption, for example, aluminum monostearate and gelatin.

Upon formulation, solutions will be administered in a manner compatible with the dosage formulation and in such amount as is therapeutically effective. The formulations are easily administered in a variety of dosage forms, such as the type of injectable solutions described above. It is contemplated that slow release capsules, timed-release microparticles, and the like can also be employed. These particular aqueous solutions are especially suitable for intravenous, intramuscular, subcutaneous and intraperitoneal administration.

The active therapeutic agents may be formulated within a mixture to comprise about 0.0001 to 1.0 milligrams, or about 0.001 to 0.1 milligrams, or about 0.1 to 1.0 or even about 1 to 10 gram per dose. Single dose or multiple doses can also be administered on an appropriate schedule for a predetermined condition.

In another embodiment, nasal solutions or sprays, aerosols or inhalants may be used to deliver the compound of interest. Additional formulations that are suitable for other modes of administration include suppositories and pessaries. A rectal pessary or suppository may also be used. In general, for suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

A pharmaceutical composition may be prepared with carriers that protect active ingredients against rapid elimination from the body, such as time-release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and others are known.

Pharmaceutical compositions are administered in an amount, and with a frequency, that is effective to inhibit or alleviate side effects of IBD. The precise dosage and duration of treatment may be determined empirically using known testing protocols or by testing the compositions in model systems known in the art and extrapolating therefrom. Dosages may also vary with the severity of the condition. A pharmaceutical composition is generally formulated and administered to exert a therapeutically useful effect while minimizing undesirable side effects

It will be apparent that, for any particular subject, specific dosage regimens may be adjusted over time according to the individual need. Doses for administration can be anywhere in a range between about 0.01 mg and about 100 mg per ml of biologic fluid of treated patient. In one particular embodiment, the range can be between 1 and 100 mg/kg which can be administered daily, every other day, biweekly, weekly, monthly etc. In another embodiment, the range can be between 10 and 75 mg/kg introduced weekly to a subject. A therapeutically effective amount of α1-antitrypsin, or drugs that have similar activities as α1-antitrypsin can be also measured in molar concentrations and can range between about 1 nM to about 2 mM.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent.

Liposomes can be used as a therapeutic delivery system and can be prepared in accordance with known laboratory techniques. In addition, dried lipids or lyophilized liposomes prepared as previously described may be reconstituted in a solution of active agent (e.g. nucleic acid, peptide, protein or chemical agent), and the solution diluted to an appropriate concentration with a suitable solvent known to those skilled in the art. The amount of active agent encapsulated can be determined in accordance with standard methods.

In each of the aforementioned compositions and methods, a compound having no significant serine protease inhibitor activity but having other α1-antitrypsin activity or analog thereof may be used in a single therapeutic dose, acute manner or a chronic manner to treat episodes or prolonged bouts, respectively, in reducing or eliminating a medical disorder contemplated herein.

In certain embodiments of the methods of the present invention, the subject may be a mammal such as a human or a veterinary and/or a domesticated animal.

### Therapeutic Methods

In one embodiment of the present invention, methods provide for treating a subject having a bowel disorder. For example, treatments for reducing the effects of an IBD or modulating progression of the disorder are contemplated herein.

Desirable blood levels may be maintained by continuous infusion to provide about 0.01-5.0 mg/kg/hr or by intermittent infusions containing about 0.4-20 mg/kg of the active ingredient(s). Buffers, preservatives, antioxidants and the like can be incorporated as required. It is intended herein that the ranges recited also include all those specific percentage amounts between the recited range. For example, the range of about 0.4 to 20 mg/kg also encompasses 0.5 to 19.9%, 0.6 to 19.8%, etc, without actually reciting each specific range therewith.

### Isolated Proteins

One embodiment pertains to isolated proteins, and biologically active portions thereof. In one embodiment, the native polypeptide can be isolated from cells or tissue sources by an appropriate purification scheme using standard protein purification techniques. In certain embodiments, the native polypeptide may be heated or otherwise treated to reduce or eliminate serine protease inhibitor activity. In certain particular embodiments, serine protease inhibitor activity is reduced where no significant activity remains. In another embodiment, polypeptides contemplated herein are produced by recombinant DNA techniques. Alternative to recombinant expression, a polypeptide can be synthesized chemically using standard peptide synthesis techniques. Any of the peptide or protein molecules contemplated of use in compositions disclosed herein can be compositions having no significant serine protease inhibitor activity. For example, AAT compositions may be treated in order to reduce or eliminate serine protease inhibitor activity or an AAT polypeptide may be isolated wherein the polypeptide has reduced or no significant serine protease inhibitor activity.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium. When the protein is produced by chemical synthesis, it is preferably substantially free of chemical precursors or other chemicals. For example, such preparations of the protein have less than about 30%, 20%, 10%, 5% (by dry weight) of chemical precursors or compounds other than the polypeptide of interest.

Compounds herein can be used as therapeutic agents in the treatment of a physiological (especially pathological) condition caused in whole or part, by excessive serine protease activity. In addition, a physiological (especially pathological) condition can be inhibited in whole or part. Peptides contemplated herein may be administered as free peptides or pharmaceutically acceptable salts thereof. Peptides may be administered to a subject as a pharmaceutical composition, which, in most cases, will include the peptide and/or pharmaceutical salts thereof with a pharmaceutically acceptable carrier.

When utilizing BLAST, Gapped BLAST, and PSI-Blast programs, the default parameters of the respective programs (e.g., XBLAST and NBLAST) can be used.

Variants of a protein contemplated herein which function as either agonists (mimetics) or as antagonists can be identified by screening combinatorial libraries of mutants, e.g., truncation mutants, of the protein of the invention for agonist or antagonist activity.

Variants of AAT molecules having no significant serine protease activity can be generated by mutagenesis, e.g., discrete point mutation or truncation. An agonist can retain substantially the same, or a subset, of the biological activities of the naturally occurring form of the protein except no significant serine protease activity remains. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity.

### Fusion Polypeptides

In other embodiments, compounds having AAT activity other than serine protease inhibitor activity, such as AAT and/or analog thereof, may be part of a fusion polypeptide. In one example, a fusion polypeptide may include AAT (e.g. mammalian α1-antitrypsin) or an analog thereof and a different amino acid sequence that may be heterologous to AAT or analog substance, having no significant serine protease inhibitor activity.

In yet other embodiments, a fusion polypeptide (e.g., IgG or fragment thereof) contemplated of use in methods herein can additionally include an amino acid sequence that is useful for identifying, tracking or purifying the fusion polypeptide, e.g., a FLAG or HIS tag sequence. The fusion polypeptide can include a proteolytic cleavage site that can remove the heterologous amino acid sequence from the compound capable of serine protease inhibition, such as mammalian AAT or analog thereof.

In one embodiment, fusion polypeptides can be produced by recombinant DNA techniques. Alternative to recombinant expression, a fusion polypeptide of the invention can be synthesized chemically using standard peptide synthesis techniques. In addition, a fusion polypeptide disclosed herein can include a pharmaceutically acceptable carrier, excipient or diluent.

### Combination Therapies

Any of the embodiments detailed herein may further include one or more a therapeutically effective amount of anti-microbial drugs, anti-inflammatory agent, immunomodulatory agent, or immunosuppressive agent or combination thereof.

Examples of anti-bacterial agents include, but are not limited to, penicillins, quinolonses, aminoglycosides, vancomycin, monobactams, cephalosporins, carbacephems, cephamycins, carbapenems, and monobactams and their various salts, acids, bases, and other derivatives.

Anti-fungal agents contemplated of use herein can include, but are not limited to, caspofungin, terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide.

Anti-viral agents contemplated of use herein can include, but are not limited to, valgancyclovir, amantadine hydrochloride, rimantadin, acyclovir, famciclovir, foscamet, ganciclovir sodium, idoxuridine, ribavirin, sorivudine, trifluridine, valacyclovir, vidarabin, didanosine, stavudine, zalcitabine, zidovudine, interferon alpha, and edoxudine.

Anti-parasitic agents contemplated of use herein can include, but are not limited to, pirethrins/piperonyl butoxide, permethrin, iodoquinol, metronidazole, diethylcarbamazine citrate, piperazine, pyrantel pamoate, mebendazole, thiabendazole, praziquantel, albendazole, proguanil, quinidine gluconate injection, quinine sulfate, chloroquine phosphate, mefloquine hydrochloride, primaquine phosphate, atovaquone, co-trimoxazole, (sulfamethoxazole/trimethoprim), and pentamidine isethionate.

Immunomodulatory agents can include for example, agents which act on the immune system, directly or indirectly, by stimulating or suppressing a cellular activity of a cell in the immune system, (e.g., T-cells, B-cells, macrophages, or antigen presenting cells (APC)), or by acting upon components outside the immune system which, in turn, stimulate, suppress, or modulate the immune system (e.g., hormones, receptor agonists or antagonists, and neurotransmitters); other immunomodulatory agents can include immunosuppressants or immunostimulants. Anti-inflammatory agents can include, for example, agents which treat inflammatory responses, tissue reaction to injury, agents which treat the immune, vascular, or lymphatic systems or any combination thereof.

Anti-inflammatory or immunomodulatory drugs or agents contemplated of use herein can include, but are not limited to, interferon derivatives, e.g., betaseron, β-interferon; prostane derivatives, iloprost, cicaprost; glucocorticoids such as cortisol, prednisolone, methylprednisolone, dexamethasone; immunosuppressive agents such as cyclosporine A, FK-506, methoxsalen, thalidomide, sulfasalazine, azathioprine, methotrexate; lipoxygenase inhibitors, e.g., zileutone, MK-886, WY-50295, SC-45662, SC-41661A, BI-L-357; leukotriene antagonists; peptide derivatives for example ACTH and analogs; soluble TNF (tumor necrosis factor)-receptors; TNF-antibodies; soluble receptors of interleukines, other cytokines, T-cell-proteins; antibodies against receptors of interleukins, other cytokines, and T-cell-proteins.

Other agents of use in combination with compositions herein can be molecules having serine protease inhibitor activity. For example serine protease inhibitors contemplated of use herein can include, but are not limited to, leukocyte elastase, thrombin, cathepsin G, chymotrypsin, plasminogen activators, and plasmin.

In certain embodiments, a composition may include one or more peptides of an AAT or AAT analog where the peptide(s) have similar activity to an AAT or AAT analog having no significant serine protease inhibitor activity. In each of the recited methods, an α1-antitrypsin (e.g. mammalian derived) substance having no significant serine protease inhibitor activity contemplated for use within methods disclosed herein can include a series of peptides including carboxyterminal or amino terminal amino acid peptides corresponding to or derived from any AAT molecule contemplated herein. In certain embodiments, the peptides can be 5 or 10 or 20 or 30 or 40 or more amino acids in length.

In addition, other combination compositions of methods disclosed herein can include certain antibody-based therapies. Non-limiting examples include, polyclonal anti-lymphocyte antibodies, monoclonal antibodies directed at the T-cell antigen receptor complex (OKT3, TIOB9), monoclonal antibodies directed at additional cell surface antigens, including interleukin-2 receptor alpha. In certain embodiments, antibody-based therapies may be used as induction therapy in combination with the compositions and methods disclosed herein.

Subjects contemplated herein can include human subjects, or other subjects such as non-human subjects, including but not limited to, primates, dogs, cats, horses, cows, pigs, guinea pigs, birds and rodents.

### Constructs of Various AAT-Related Carboxyterminal Peptides

Embodiments herein provide for generating and using recombinant AAT or recombinants having one or more carboxyterminal peptides derived from AAT (e.g. a carboxyterminal peptide of AAT found in the last 80 amino acids of AAT).

In one embodiment of the present invention, a composition may include constructs that engage molecules that bind the SEC receptor for treating a subject in need of AAT therapy (e.g. mammalian derived AAT) for example, a series of peptides including carboxyterminal amino acid peptides corresponding to AAT. These peptides can include, pentapetides including, FVFLM (SEQ ID NO:2), FVFAM (SEQ ID NO:3), FVALM (SEQ ID NO:4), FVFLA (SEQ ID NO:5), FLVFI (SEQ ID NO:6), FLMII (SEQ ID NO:7), FLFVL (SEQ ID NO:8), FLFVV (SEQ ID NO:9), FLFLI (SEQ ID NO:10), FLFFI (SEQ ID NO:11), FLMFI (SEQ ID NO:12), FMLLI (SEQ ID NO:13), FIIMI (SEQ ID NO:14), FLFCI (SEQ ID NO:15), FLFAV (SEQ ID NO:16), FVYLI (SEQ ID NO:17), FAFLM (SEQ ID NO:18), AVFLM (SEQ ID NO:19), and any combination thereof.

In other embodiments, AAT peptides contemplated for use in constructs, pharmaceutical compositions and methods herein are also intended to include any and all of those specific AAT peptides of SEQ ID NO:1 (naturally-occurring AAT of 394 amino acids, the most common form is the M type with subtypes M1, M2, M3 etc. are also contemplated herein) associated with the carboxyterminal 80 amino acids. All AAT polypeptides are contemplated of use in methods disclosed herein, that possess anti-inflammatory activity and/or immune regulatory activity. Any combination of consecutive amino acids simulating AAT or AAT-like activity may be used, such as amino acids ranging from 315-394, amino acids ranging from 325-384, 340-380 etc. In addition, combinations of consecutive amino acid sequences such as 5-mers, 10-mers, 15-mers, 20-mers etc. of the carboxyterminus can also be used. For example, any combinations of consecutive amino acids of 5-mers, 10-mers, 15-mers, 20-mers from SEQ ID NO:1 AAs 314-394 can be used in developing or purifying a construct contemplated herein.

In some embodiments, AAT protease binding domain can be mutated in order to reduce or eliminate the protease function of the molecule and not inhibit elastase activity; these molecules can be used in any construct contemplated herein. In certain embodiments, a mutated AAT can be used to generate an AAT construct by methods disclosed herein. In other embodiments, a mutated molecule (e.g. having reduced or essentially no protease activity) retains its anti-inflammatory effects and/or immunomodulatory effects and can be used as an anti-inflammatory molecule in a subject having a need for AAT therapy. One skilled in the art would understand a non-protease binding domain of AAT as well as what is termed the carboxyterminal last 80 amino acids.

In each of the above-recited methods, α1-antitrypsin or carboxyterminal peptide derivatives thereof are contemplated for use in a composition herein. These peptide derivatives may include but are not limited to amino acid peptides containing the last 80 carboxyterminal derived amino acids of AAT, GITKVFSNGA (SEQ ID NO:20), DLSGVTEEAP (SEQ ID NO:21), LKLSKAVHKA (SEQ ID NO:22), VLTIDEKGTE (SEQ ID NO:23), AAGAMFLEAI (SEQ ID NO:24), PMSIPPEVKF (SEQ ID NO:25), NKPFVFLMIE (SEQ ID NO:26), QNTKSPLFMG (SEQ ID NO:27), KVVNPTQK (SEQ ID NO:28), LEAIPMSIPPEVKFNKPFVFLM (SEQ ID NO:29); and LEAIPMSIPPEVKFNKPFVF (SEQ ID NO:30), GADLSGVTEEAPLKLSKAVHKAV LTIDEKGTEAAGAMFLERIPV SIPPEVKFNKPFVFLMIEQNTKSPLFMGKVVNPTQK (SEQ ID NO:31) or any combination thereof.

In certain embodiments, compositions of recombinant AAT or AAT-derived carboxyterminal peptides capable of binding to SEC receptors or compositions with AAT-like activities may be administered to a subject in need thereof. As disclosed herein the carboxyterminal region of AAT includes the last 80 amino acids of SEQ ID NO:31 or human AAT molecule or other naturally occurring AAT molecule. In other embodiments, peptides derived from AAT can include 5-mers, 10-mers, 20-mers, 25-mers, 30-mers, 35-mers, 40-mers, 50-mers, and up to an 80-mer of an AAT molecule wherein any of the contemplated peptides have no significant serine protease inhibitor activity, are derived from the carboxyterminus of AAT and are capable of being used for treating subjects undergoing radiation or subjects exposed to large doses of radiation by accident or other cause.

In one embodiment of the present invention, a construct may include compounds that engage or associate with the SEC receptor. In some of the recited methods, an AAT-mutant or AAT derived peptide (e.g. mammalian derived) having no significant serine protease inhibitor activity contemplated for use within the methods of the present invention can include a series of peptides including carboxyterminal amino acid peptides corresponding to AAT. In addition, combinations of amino acid 5-mers or 10-mers or 20-mers or 30-mers or more can also be used. For example, one or more 5-mers or 10-mers or 20-mers etc can include consecutive amino acids starting from AA 315 and ending with AA 394 of naturally occurring AAT represented as SEQ ID NO:31. As contemplated herein, the later half of a sequence toward the carboxy end is referred to as the carboxyterminus. In certain embodiments, the carboxyl domain of AAT going backwards from the carboxyl terminus is defined as those amino acids most conserved among the difference species and do not participate in the protease binding domain of AAT. In addition, in other embodiments, AAT protease binding domain can be mutated in order to reduce or eliminate the protease function of the molecule and this molecule can be used in any composition contemplated herein. In other embodiments, a mutated molecule can retain its anti-inflammatory and/or immunomodulatory effects. Also contemplated herein is that the carboxyl domain is the non-protease binding domain. One skilled in the art would understand a non-protease binding domain of AAT.

### AAT

In each of the above-recited methods, compositions herein may include peptides derived from the carboxyterminus of AAT. In certain embodiments, AAT-associated molecules used in the methods and compositions herein can include, but are not limited to, compositions of SEQ ID NO:1, naturally occurring AAT (394 AA length molecule making up approximately 90% of AAT isolated from serum), other AAT M-types or other AAT molecules.

Human AAT is a single polypeptide chain with no internal disulfide bonds and only a single cysteine residue normally intermolecularly disulfide-linked to either cysteine or glutathione. One reactive site of AAT contains a methionine residue, which is labile to oxidation upon exposure to tobacco smoke or other oxidizing pollutants. Such oxidation reduces the elastase-inhibiting activity of AAT; therefore substitution of another amino acid at that position, e.g., alanine, valine, glycine, phenylalanine, arginine or lysine, produces a form of AAT which is more stable. Native AAT can be represented by the following sequence:

(SEQ ID NO: 1)
EDPQGDAAQKTDTSHHDQDHPTFNKITPNLAEFAFSLYRQLAHQSNSTNIFFSPVS
IATAFAMLSLGTKADTHDEILEGLNFNLTEIPEAQIHEGFQELLRTLNQPDSQLQLTTGN
GLFLSEGLKLVDKFLEDVKKLYHSEAFTVNFGDTEEAKKQINDYVEKGTQ
GKIVDLVKELDRDTVFALVNYIFFKGKWERPFEVKDTEEEDFHVDQVTTVKVPMMKRLGM
FNIQHCKKLSSWVLLMKYLGNATAIFFLPDEGKLQHLENELTHDIITKFLENEDRRSASL
HLPKLSITGTYDLKSVLGQLGITKVFSNGADLSGVTEEAPLKLSKAVHKAVLTIDEKGTE
AAGAMFLEAIPMSIPPEVKFNKPFVFLMIEQNTKSPLFMGKVVNPTQK.

Other sequences exist with some variability.

Extra hepatic sites of AAT production include neutrophils, monocytes and macrophages, and the expression of AAT is inducible in response to LPS, TNFα, IL-1 and IL-6 in various cell types. Deficiency in AAT is associated with immune dysfunctional conditions such as rheumatoid arthritis and systemic lupus erythematosus.

In one embodiment, with respect to the use of the compositions and methods of the present invention specifically excluded within the scope of the present invention are those furin endoprotease inhibitors comprising an AAT variant having an amino acid sequence comprising the amino acids of the native AAT molecule, except that the sequence at position 355-358 of the native protein (-Ala-Ile-Pro-Met-) is changed to the novel sequence -Arg-X-X-Arg-, wherein X is any amino acid, at positions 355-358 of the native α1-antitrypsin amino acid sequence as disclosed in U.S. Pat. Nos. 5,604,201 and 6,022,855.

Also specifically excluded within the scope of the compositions and methods with respect to the use of the compositions and methods of the present invention AAT Portland variants wherein the amino acid sequence at positions 355-358 of the AAT Portland sequence is -Arg-Ile-Pro-Arg- as disclosed in U.S. Pat. Nos. 5,604,201 and 6,022,855.

Also specifically excluded within the scope of the compositions and methods are AAT are peptides having amino acid sequences comprising the amino acid sequence -Arg-Xaa-Xaa-Arg- at positions 355-358, wherein each Xaa is any amino acid as is disclosed in U.S. Pat. Nos. 5,604,201 and 6,022,855.

### Kits

In still further embodiments, kits for use with the methods described above are contemplated. Kits may include AAT, one or more carboxyterminal-derived AAT molecules, a mutant AAT composition, a mutant AAT molecule associated with a gene therapy delivery system or other combinations for treatment of one or more inflammatory bowel diseases. In addition, other agents such as anti-bacterial agents, immunosuppressive agents, anti-inflammatory agents may be provided in the kit. The kits can include, a suitable container means, composition disclosed herein, optionally, a delivery instrument and optionally, one or more additional agents.

The kits may further include a suitably aliquoted composition of the encoded protein or polypeptide antigen, whether labeled or unlabeled, as may be used to prepare a standard curve for a detection assay.

The container means of the kits will generally include at least one vial, test tube, flask, bottle, syringe or other container means. A kit will also generally contain a second, third or other additional container into which other combination agents may be placed. Such containers may include injection or blow-molded plastic containers into which the desired vials are retained.

In certain embodiments, a kit can include a composition including, but not limited to, AAT, AAT fragment, or an AAT analog or polypeptide, having no significant serine protease inhibitor activity. In accordance with these embodiments, a kit can contain AAT or an analog thereof having no significant serine protease inhibitor activity. In some embodiments, a kit may be provided for a health provider. In other embodiments, a kit may be provided for a patient for home or portable use.

### EXAMPLES

The following examples are included to illustrate various embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes may be made in the specific embodiments which are disclosed and still obtain a like or similar result without departing from the spirit and scope of the invention.

### Example 1

### DSS Colitis was Significantly Attenuated by AAT Administration

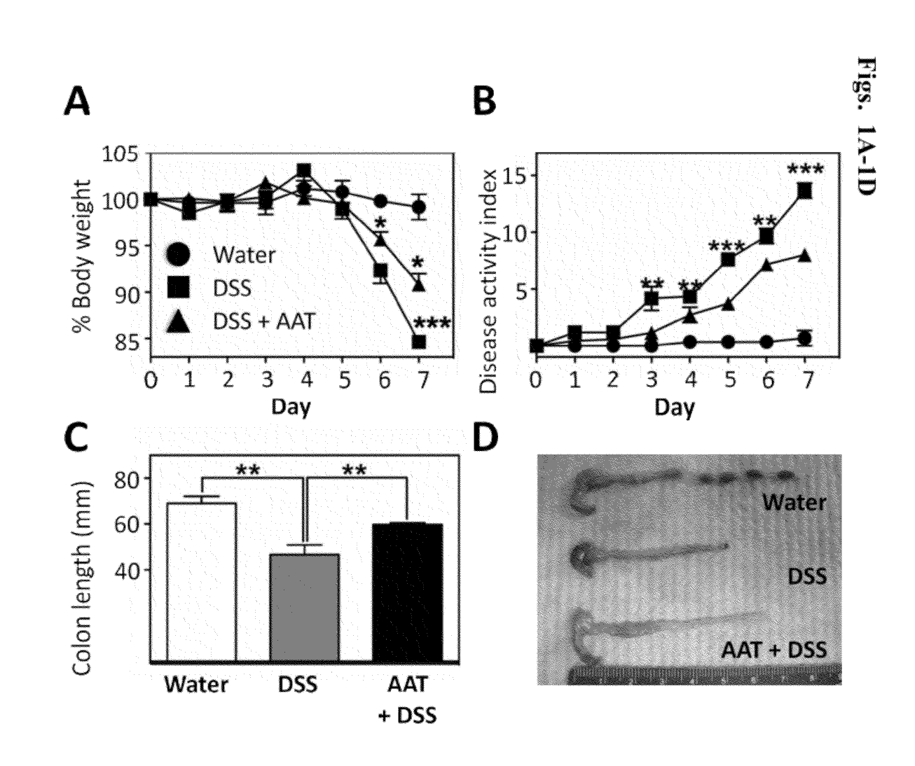

Mice treated with AAT (2 mg/day i.p) displayed significantly less weight loss in response to DSS administration compared with vehicle treated mice with weights at time of sacrifice reduced to 84.6±0.7% of initially body weight in vehicle-treated mice compared to 90.8±1.1% in AAT-treated mice (P<0.001; FIG. 1A). This coincided with reduced a disease activity index from 13.7±0.6 in vehicle-treated mice to 8.0±0.6 in AAT-treated mice (P<0.001; FIG. 1B) and decreased colon shortening from 46.7±4.2 mm in vehicle mice to 59.7±0.8 mm in AAT-treated mice (P<0.01; FIG. 1C). This significant attenuation of colitis is sufficiently potent to be visible at a macroscopic level also (D).

FIG. 1 represents alpha 1 antitrypsin attenuated acute colitis in mice. Concomitant administration of AAT during DSS insult resulted in a significant attenuation of weight loss (A), reduced disease severity (B) and decreased colon shortening (C) compared to vehicle treated mice (Mean SEM for N=6 mice/group; *P<0.05, **P<0.01 ***P<0.001). This attenuation of inflammation and colon shortening was clearly visible macroscopically as shown by the representative image of colons excised from mice of each treatment group (D).

### Example 2

### Cytokine Production by Colonic Explants was Decreased in AAT-Treated Mice Compared with Vehicle-Treated DSS Mice

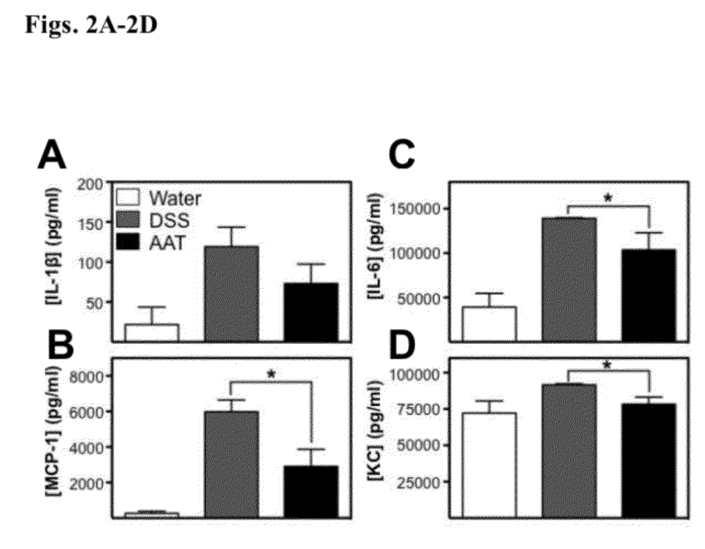

Release of IL-1β from colonic explants was significantly increased in DSS treated mice compared with water treated (21.8±2.1 pg/ml in Water to 134.3±23.6 pg/ml in DSS; P<0.01), whereas in mice treated with AAT, levels of IL-1β were not significantly increased compared to water control mice (73.1±24.4 pg/ml; p=0.1; FIG. 2). The concentrations of IL-6 from 24 hr culture supernatants of DSS colonic explants were significantly elevated compared with water controls (39.2±15.5 ng/ml in water-treated mice to 139.1±0.7 ng/ml in DSS; P<0.0001), which was significantly reduced by administration of AAT (103.6±19.3 ng/ml; P<0.05). MCP-1 liberated from DSS colitis explants was significantly higher than from water control mice (259.4±122.1 pg/ml to 5981±666 pg/ml; P<0.0005) and once again this was significantly decreased in AAT-treated DSS colitic mice (3390±991.8 pg/ml; P<0.05). DSS colitis increased release of KC by colonic explants (91.6±0.7 ng/ml in vehicle; P<0.01) compared to water alone, however, this increase was significantly reduced by treatment with AAT (78.3±4.9 ng/ml; N=6; P<0.05) to levels comparable to water treatment (72.2±8.3 ng/ml). It was observed that there were no significant effects of AAT on release of IL-10 or IL-17A.

FIG. 2 represents reduced cytokine release from colonic explants of AAT-treated mice compared with vehicle treated controls. Assessment of cytokine released into supernatants of colic tissue explants following 24 hr culture demonstrated that IL-1, IL-6, MCP-1 and KC were all significantly increased in DSS colitic mouse tissues compared with water treated mice. Treatment of DSS mice with AAT reduced concentrations of IL-1 to levels comparable with water-treated mice while also significantly reducing release of IL-6, MCP-1 and KC compared with vehicle-treated DSS mice.

### Example 3

### Alpha 1 Anti-Trypsin Reduced CD4+ T Lymphocyte Accumulation in the Colonic Lamina Propria During DSS Colitis

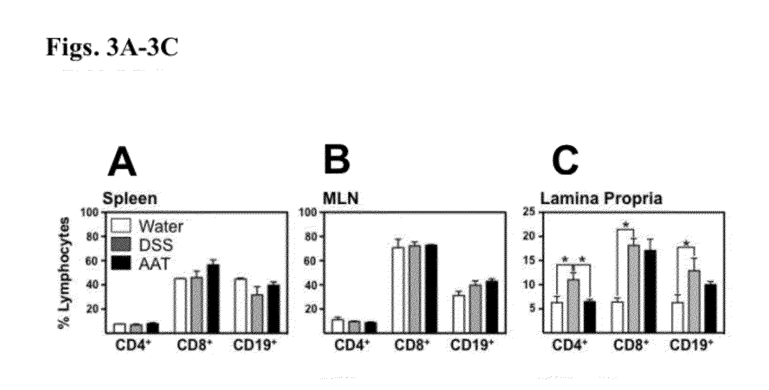

Subset analysis of leukocytes from the spleen, MLN and colonic lamina propria of mice administered water or DSS with or without AAT 2 mg/mouse/day by i.p. injection demonstrated no change in proportion of cell types analyzed between DSS and DSS+AAT in the spleen. In contrast, in the draining MLN there was a significant decrease in the proportion of macrophages (37.7±0.9 in DSS to 31.7±1.3 AAT-treated mice; P<0.01) and neutrophils (19.0±0.6 in DSS to 13.6±0.8 in AAT-treated mice; P<0.001). Furthermore in the colonic lamina propria there was a significant decrease in the proportion of CD4+ T lymphocytes (11.4±1.4 in DSS to 6.5±0.5 in AAT-treated mice; P<0.05; FIG. 3).

FIG. 3 represents that a proportion of CD4+ T lymphocytes was decreased by AAT treatment in DSS mice compared with vehicle controls. The percentages of CD4+ (CD4 T cells), CD8+ (CD8 T cells), CD19+ (B cells), CD11c+MHCII++F4/80-(dendritic cells), F4/80+ (macrophages), GR-1+ (neutrophils), Siglec F+ (eosinophils), NK1.1+ (NK cells) and, NK1.1+CD3+ (NKt cells) from indicated organs of water-treated, DSS or DSS & AAT-treated mice were analyzed by flow cytometry. While no significant changes were observed in the spleen or MLN, there was a significant increase in CD4+, CD8+ and CD19+ lymphocytes in the lamina propria of DSS-treated mice compared with water controls (P<0.05). Furthermore, treatment with AAT significantly reduced the proportion of CD4+ T cells in DSS colitis compared to vehicle-treated mice (P<0.05). Results represent mean±SEM for N 3 mice per treatment. P<0.05.

### Example 4

### Mucosal Healing Expedited by Treatment with AAT Following DSS Colitis

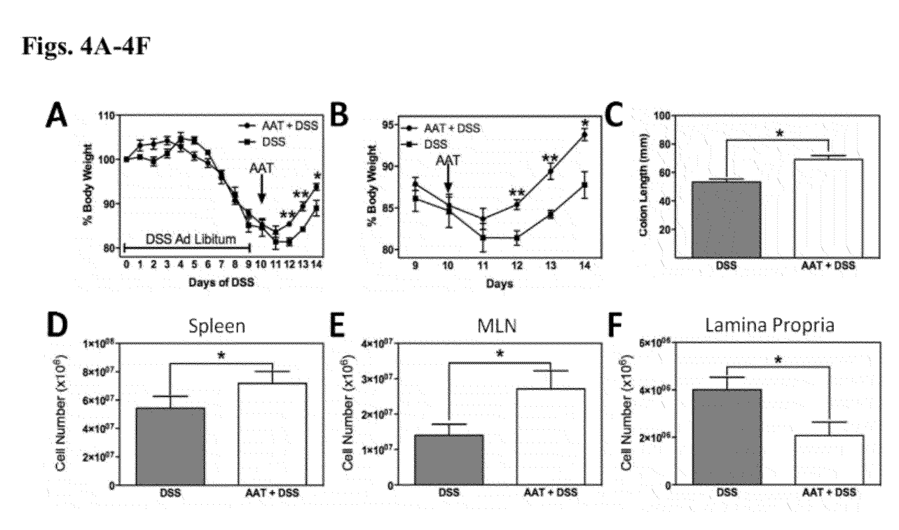

Following the injury of the colonic mucosa of mice treated with 3% DSS and the concomitant weight loss of 15%, mice received water ad libitum and the recovery of body weight was assessed (FIG. 4A). Treatment with AAT (5 mg) significantly increased the weight of mice throughout the 4 d recovery period with the most significant difference on day 13 when the difference in mean weight loss between treatment groups differed from 10.3±0.9 in AAT-treated mice to 15.6±0.6 in vehicle treated mice (N=5; P<0.01; FIG. 4B). The increased weight gain by AAT-treated mice coincided with a significant reduction in colon shortening from 53.2±2.2 mm in vehicle-treated mice to 69.0±2.8 mm in AAT-treated mice (N=5; P<0.05; FIG. 4C). Quantification of cells from DSS-treated mice demonstrated that mice treated with AAT had a significant increase in the total numbers of cells in the spleen compared with from 5.4×107±8.5×106 to 7.2×107±8.4×106 (P<0.05; N=5, FIG. 4D) and MLN from 1.4×107±3.1×106 in vehicle-treated mice to 2.7×107±5.1×106 in AAT-treated mice (P<0.05; FIG. 4E). In contrast, there was a significant decrease in inflammatory infiltrate in the colonic lamina propria of DSS-treated mice from 4.0×106±5.2×105 in vehicle-treated mice to 2.1×106±5.6×105 in AAT-treated mice (P<0.05; FIG. 4F).

FIG. 4 represents AAT expedited recovery following withdrawal of DSS insult. Administration of AAT following DSS insult resulted in a significant recovery of lost weight (A, B), reduced colon shortening (C), accumulation of cells in the spleen and MLN (D, E) as well as reduced colonic inflammatory infiltrate in AAT-treated mice compared to vehicle treated mice (Mean SEM for N=6 mice/group; *P<0.05, **P<0.01 ***P<0.001).

### Example 5

### Altered Lymphocyte Populations in the Recovering Colon Due to AAT-Treatment

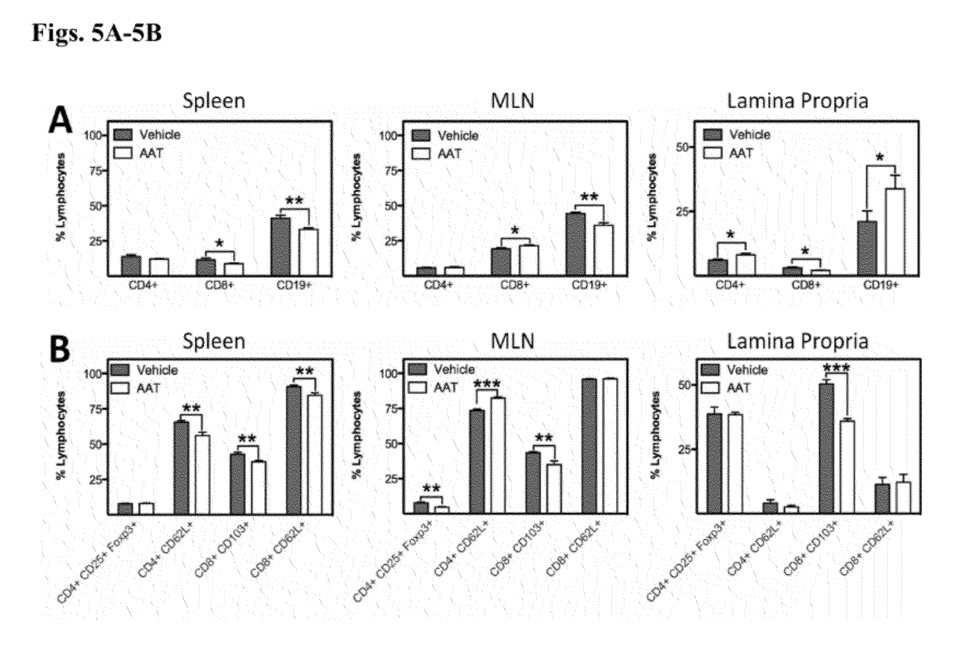

Analysis of CD4, CD8 and CD19 T cells demonstrated that in the spleen, the proportion of CD8+ in AAT-treated mice (8.9±0.3) compared with vehicle-treated mice (11.6±1.3; P<0.05) and CD19+ cells from AAT-treated mice (33.2±1.1) compared to vehicle treated mice (41.2±2.0; P<0.05) were significantly decreased (FIG. 5A). In the MLN, there was a significant decrease in the proportion of CD8+ cells from AAT-treated (21.5±0.6) to vehicle-treated mice (19.3±0.7) while the percentage of CD19+ cells increased in AAT-treated mice (36.0±1.7) compared to vehicle-treated mice (44.4±0.9; P<0.05; FIG. 5A). In the lamina propria, there was a significant decrease in CD4+ from AAT-treated mice (8.1±0.6) to vehicle-treated mice (6.1±0.4) and CD19+ cells from AAT-treated mice (33.9±5.2) to vehicle-treated mice (21.1±4.1) while the proportion of CD8+ cells increased significant (from 2.1±0.1 to 3.1±0.3; P<0.05). Subsequent analysis of the T cell subsets revealed a significant decrease in CD4+CD62LHigh naïve T cells from AAT-treated mice (56.1±2.4) to vehicle-treated mice (65.5±1.3) and CD8+CD62LHigh naïve T cells from AAT-treated mice (84.5±1.8) to vehicle-treated mice (90.7±1.0) as well as regulatory CD8+CD103+ T cells from AAT-treated mice (37.5±0.9) to vehicle-treated mice (42.9±1.3) in the spleen (P<0.01; FIG. 5B). In the MLN there was a significant decrease regulatory CD4+CD25+Foxp3+ from AAT-treated mice (4.7±0.5) compared with vehicle-treated mice (7.8±0.6) and CD8+ CD103+ T cells from AAT-treated mice (35.0±2.6) compare with vehicle-treated mice (44.8±1.6), though naïve CD4 T cells were increased from AAT (82.4±0.8) to vehicle-treated mice (73.5±0.9; P<0.001; FIG. 5B). In the lamina propria there was a significant decrease in regulatory CD8+ CD103+ T cells from AAT-treated mice (35.90±1.070 N=4) to vehicle-treated mice (50.4±1.7; P<0.001; FIG. 5B). Results represent mean±SEM for N≧3 mice per treatment.

### Example 6

### Differential Leukocytes Expression in DSS Recovery Colons with AAT-Treatment

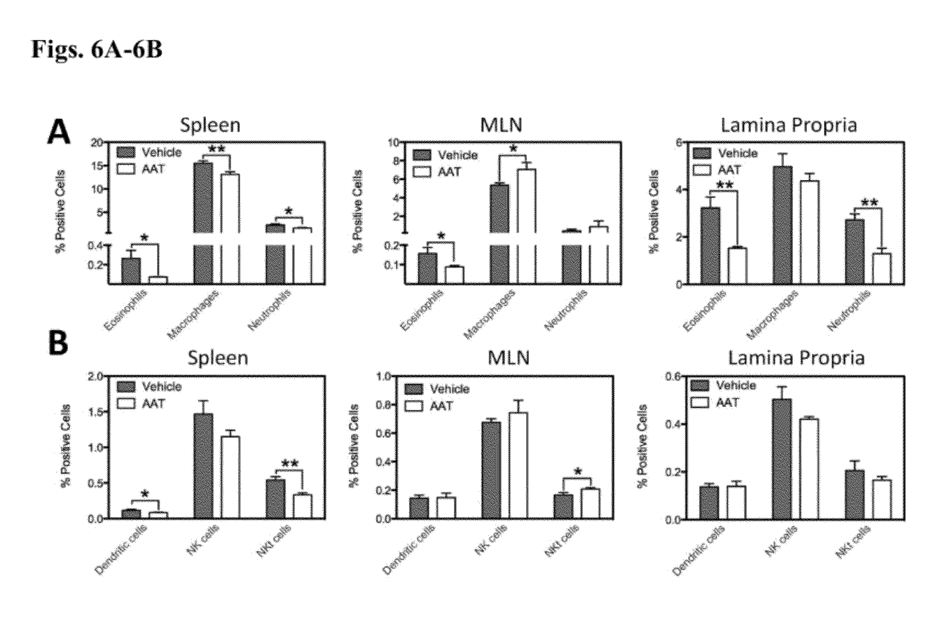

The proportion of eosinophils, macrophages, neutrophils, dendritic cells and NKt cells were all significantly decreased in the spleen of AAT-treated mice compared with vehicle control mice. Eosinophils were also decreased in the MLN, in contrast to an increase in both macrophages and NKt cells (P<0.05; FIGS. 6A&B). In the colonic lamina propria there was a significant decrease in eosinophils and neutrophils while other populations were decreased uniformly. More detailed comparison of tissue and cell type is described below. Results represent mean±SEM for N≧3 mice per treatment.

FIG. 6 represents Siglec F+ (eosinophils), F4/80+ (macrophages), GR-1+ (neutrophils), CD11c+MHCII++F4/80-(dendritic cells), NK1.1+ (NK cells) and, NK1.1+ CD3+ (NKt cells) from indicated organs of water-treated, DSS or DSS & AAT-treated mice were analyzed by flow cytometry. The proportion of eosinophils, macrophages, neutrophils, dendritic cells and NKt cells were all significantly decreased in the spleen of AAT-treated mice compared with vehicle control mice. Eosinophils were also decreased in the MLN, in contrast to an increase in both macrophages and NK t cells (P<0.05; FIGS. 6A&B). In the colonic lamina propria there was a significant decrease in eosinophils and neutrophils while other populations were decreased uniformly. Results represent mean±SEM for N 3 mice per treatment. *P<0.05, **P<0.01

TABLE 1

| | DSS + Vehicle | | | DSS + AAT | | |
|---|---|---|---|---|---|---|
| | Spleen | MLN | LP | Spleen | MLN | LP |
| Eosinophils | 0.3 ± 0.1 | 0.1 ± 0.0 | 1.5 ± 0.1 | 0.1 ± 0.0 | 0.2 ± 0.0 | 3.2 ± 0.5 |
| (P value) | (0.04) | (0.03) | (0.002) | | | |
| Macrophages | 13.1 ± 0.6 | 7.1 ± 0.7 | 4.4 ± 0.3 | 15.5 ± 0.5 | 5.4 ± 0.3 | 5.0 ± 0.6 |
| (P value) | (0.006) | (0.03) | (0.2) | | | |
| Neutrophils | 1.6 ± 0.1 | 0.9 ± 0.6 | 1.3 ± 0.2 | 2.3 ± 0.2 | 0.4 ± 0.2 | 2.7 ± 0.2 |
| (P value) | (0.02) | (0.2) | (0.001) | | | |
| Dendritic | 0.1 ± 0.0 | 0.1 ± 0.0 | 0.1 ± 0.0 | 0.1 ± 0.0 | 0.1 ± 0.0 | 0.1 ± 0.0 |
| Cells | (0.03) | (0.5) | (0.5) | | | |
| (P value) | | | | | | |
| NK cells | 1.2 ± 0.1 | 0.7 ± 0.1 | 0.4 ± 0.0 | 1.5 ± 0.2 | 0.7 ± 0.0 | 0.5 ± 0.1 |
| (P value) | (0.03) | (0.2) | (0.1) | | | |
| NKt cells | 0.3 ± 0.0 | 0.2 ± 0.0 | 0.2 ± 0.0 | 0.5 ± 0.0 | 0.2 ± 0.0 | 0.2 ± 0.0 |
| (P value) | (0.003) | (0.03) | (0.2) | | | |

Acceptable mouse model for IBS: Originally developed as a model of senescence, senescence-accelerated mouse prone 1 or SAMP1 mice develop spontaneous, segmental, transmural ileitis (1) recapitulating many features of Crohn's disease (CD). Furthermore, the idiopathic and polygenic origin of ileitis in these mice also contributes to its value in modeling human CD.

Unlike CD, ulcerative colitis, while also an inflammatory bowel disease is characterized by superficial fissuring ulceration of the colonic mucosa with little evidence of inflammatory infiltration of the underlying muscularis. The DSS model of chemically induced colitis is therefore a commonly adopted system to model the inflammatory insult associated with flaring ulcerative colitis. This model can be used to address the effect of the initial flaring insult but also the intestinal response that drives the mucosal healing process but studying mice during the recovery phase. The combination of these models, allows for the study of ileal and colonic inflammation as well as acute and chronic intestinal immune responses.

### Example 7

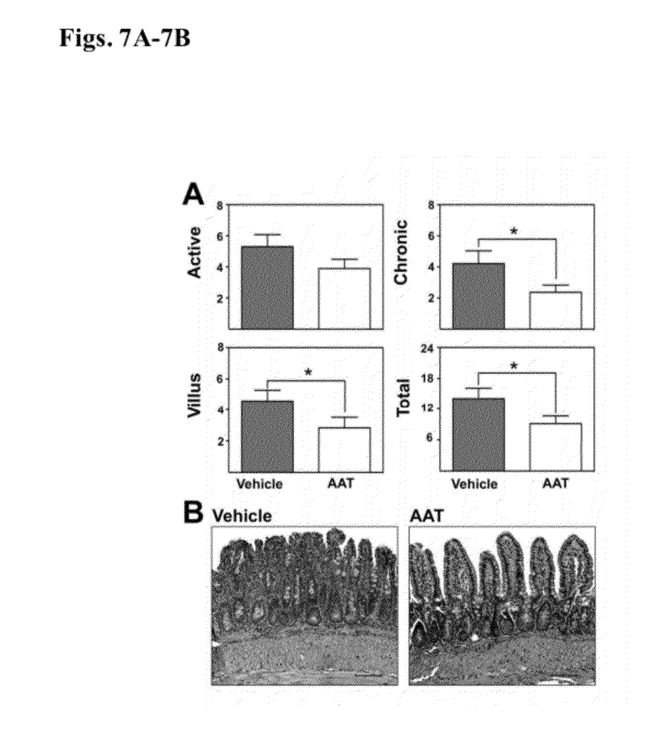

FIG. 7 represents treatment with AAT attenuated inflammation in SAMP1 mice compared with vehicle treated control littermates. A) Treatment of 20-week-old SAMP1 mice with AAT i.p. daily for 9 days did not significantly alter active inflammatory index (i.e. infiltrating granulocytes), but significantly attenuated all other assessed parameters of inflammation including lymphocyte/monocytes infiltration (chronic) and villus distortion (villus)). Total histological score represents the sum of the three separate inflammatory indices as previously described. Mean SEM for N 6 mice/group; P<0.05. B) Representative micrographs showing attenuation of inflammation by AAT treatment in the ileum of 20-week-old SAMP1 mice compared with vehicle treated controls.

### Example 8

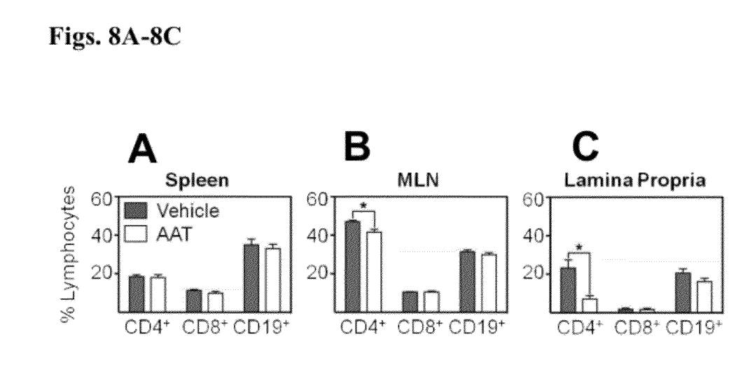

FIG. 8 represents AAT decreased the proportion of CD4+ T lymphocytes in the ileum and MLN of SAMP1 mice compared to vehicle-treated littermate controls. The percentages of CD4+ (CD4 T cells), CD8+ (CD8 T cells), CD 19+ (B cells), CD11c+MHCII++F4/80-(dendritic cells), F4/80+ (macrophages), GR-1+ (neutrophils), Siglec F+ (eosinophils), NK1.1+ (NK cells) and, NK1.1+ CD3+ (NKt cells) from indicated organs were analyzed by flow cytometry. The only population to change significantly was the CD4+ T cell population which was reduced by AAT treatment from 47.0 0.8% to 41.5 1.5% in the MLN (P<0.05) and 23.2 4.2% to 7.1 1.8% in the lamina propria (P<0.05). Results represent mean±SEM for N=4 mice per treatment. *P<0.05.

### Example 9

### Alpha-1 Antitrypsin Decreases Intestinal Permeability in the SAMP1/YitFC Model of Chronic Murine Ileitis in Vivo

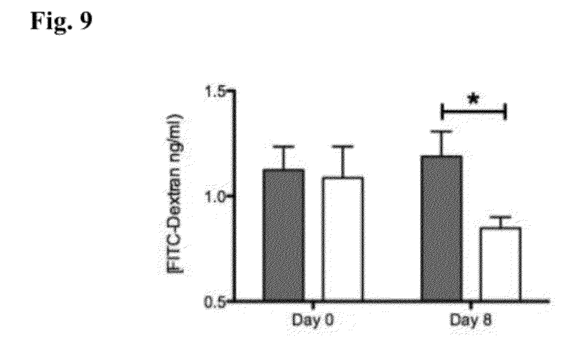

FIG. 9 represents mice having received oral gavage of fluorescently-conjugated dextran sugar (4000 mw) on day 0 and day 8 of treatment with 1 mg/mouse/day of Alpha-1 antitrypsin (AA). Serum samples were taken 4 hrs later and examined for Fitc-dextran using a fluorimeter. Results demonstrate a significant decrease in serum Fitc-dextran, a surrogate marker of intestinal barrier function on day 8 of AAT treatment.

### Example 10

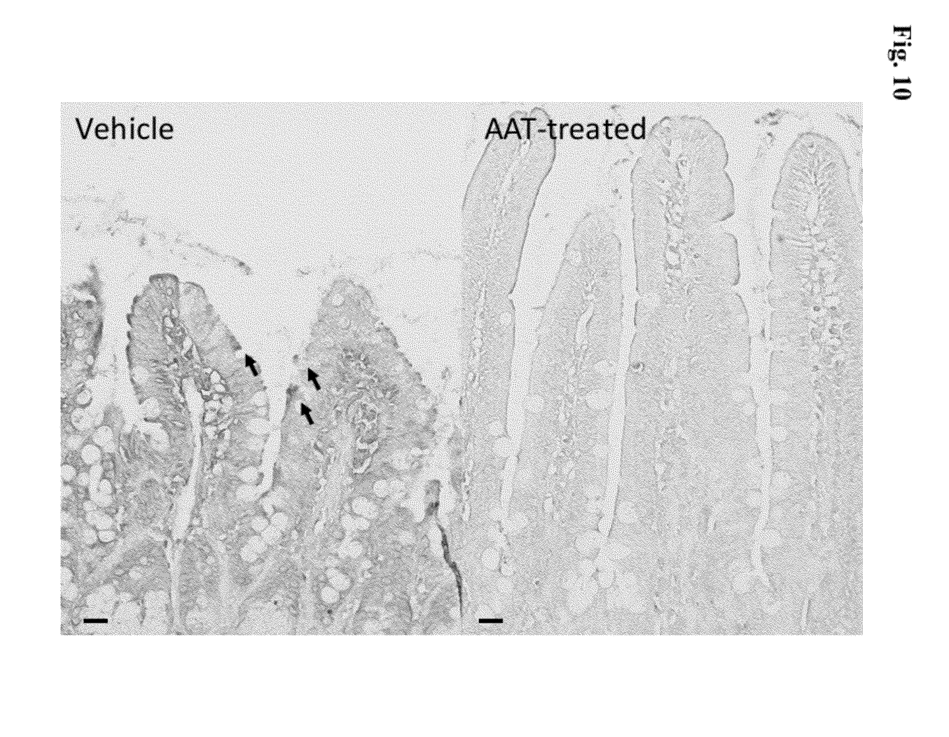

FIG. 10 represents a TUNEL staining of formalin-fixed paraffin embedded 4 μm sections from vehicle and AAT-treated 20-week-old SAMP1/YitFc. Micrographs were photographed at 20× magnification with black scale bars equal to 100 μm. Specific staining of apoptotic epithelial cells are indicated by black arrows. Sections were counterstained with methyl green for clarity. It is demonstrated that AAT has protective and restorative effects on the intestinal lining related to recovery of intestinal/bowel function in an affected subject described herein.

### Methods

Mice: C57BL6/J mice were obtained from the Jackson Laboratories (Bar Harbor, Me.) and bred in house for DSS colitis experiments. SAMP1/skuslc mice were purchased (e.g. SLC Inc, Hamamatsu Japan).

Chemically-induced acute murine colitis model: Mice were treated with DSS ad libitum (3% w/v; MP biomedicals, USA; 36-50 kDa) in drinking water for 7 days. Water alone was used for vehicle groups. DSS groups received i.p. injections of either 2 mg AAT or human serum albumin per mouse. Body weight, stool consistency and occult bleeding was assessed daily to construct a disease activity index. At the time of sacrifice, spleen, mesenteric lymph nodes and segments of colonic tissue were excised for flow cytometric analysis of leukocyte subsets, colon lengths were assessed and colon sections processed for histology, RNA extraction and cytokine analysis to assess disease activity. DSS recovery from colitis model

Mice were treated with DSS ad libitum (3% w/v; MP biomedicals, USA; 36-50 kDa) in drinking water for 9 d, replacing with fresh DSS every three days. Once mice had reached the desired target weight loss (15%) the DSS was withdrawn and replaced with water. Subsequent recovery of weight was recorded daily and used as a surrogate indicator of mucosal healing.

### Spontaneous Chronic Murine Ileitis Model

Twenty-week-old SAMP1 mice were treated for 9 days with 2 mg of AAT per mouse per day by i.p. injection. Terminal ileal tissues were harvested for RNA isolation, histological evaluation and leukocyte isolation.

### Lymphocyte and Epithelial Cell Isolation

Single-cell suspensions were obtained by gently pressing the mesenteric lymph node (MLN) or spleen against a 100 μm cell-strainer. Splenic red blood cells were lysed by 3 min incubation in ammonium chloride lysing reagent (ACK Lysis Buffer, Invitrogen, Carlsbad, Calif.). Intestinal segments were opened along the mesentery and rinsed of luminal contents with PBS before cutting into 1 cm sections in PBS containing 15 mM HEPES and 1 mM EDTA with vigorous agitation on a vortex mixer. The tissue was then passed through a 70 μm tissue strainer and the process repeated until the wash remained clear. The remaining lamina propria (LP) was digested in 1 mg/ml Collagenase Type VIII (Sigma Aldrich, C9722) for 20 min in an orbital shaker at 270 rpm and 37° C. Tissues were vortexed briefly and filtered to remove any remaining undigested material and cells counted prior to flow cytometric evaluation.

### Flow Cytometry

Cells from indicated compartments were incubated with fluorescent rat anti-mouse antibodies including against: mouse CD4 (RM4-5), CD8 (53-6.7), CD19 (1D3), Nk1.1 (PK136), CD11c (N418), MHCII (M5/114.15.2), F4/80 (BM8), Siglec F (E50-2440) and GR-1 (RB6-8C5) or their respective isotype controls. FoxP3 staining was performed according to manufacturer's instructions using the PE-FoxP3 labeling kit (eBiosciences, San Diego, Calif.). Cells were washed and fixed with 2% paraformaldehyde and analyzed using the FACS® Canto system (Beckton-Dickinson Immunocytometry Systems, San Jose, Calif.). Post-analyses were performed using FLOWJo software (Tree Star Inc, Ashland, Oreg.).

### Cytokine Production Assays

Tissue explants (0.5 cm2) were cultured for 24 h in DMEM (without sodium pyruvate, Cellgro Manassas, Va., supplemented with 5% FBS, 2 mM glutamine, 100 IU penicillin and 100 μg/ml streptomycin; Invitrogen) culture supernatants were then analyzed for the presence of cytokines using the Quansys Cytokine Assay system (Logan, Utah).

### Statistics

Statistical analyses were performed using Student t test or two-way ANOVA with Bonferroni posttests with Graphpad Prism Data Analysis software (GraphPad Software, La Jolla, Calif.). Data were expressed as mean±standard error of the mean (SEM). Statistical significance was set at a P value of less than 0.05.

DSS Recovery from Colitis Model

Mice were treated with DSS ad libitum (3% w/v; MP biomedicals, USA; 36-50 kDa) in drinking water for 9 d, replacing with fresh DSS every three days. Once mice had reached the desired target weight loss (15%) the DSS was withdrawn and replaced with water. Subsequent recovery of weight was recorded daily and used as a surrogate indicator of mucosal healing.

### Spontaneous Chronic Murine Ileitis Model

Twenty-week-old SAMP1 mice were treated for 9 days with 2 mg of AAT per mouse per day by i.p. injection. Terminal ileal tissues were harvested for RNA isolation, histological evaluation and leukocyte isolation.

### Lymphocyte and Epithelial Cell Isolation

Single-cell suspensions were obtained by gently pressing the mesenteric lymph node (MLN) or spleen against a 100 μm cell-strainer. Splenic red blood cells were lysed by 3 min incubation in ammonium chloride lysing reagent (ACK Lysis Buffer, Invitrogen, Carlsbad, Calif.). Intestinal segments were opened along the mesentery and rinsed of luminal contents with PBS before cutting into 1 cm sections in PBS containing 15 mM HEPES and 1 mM EDTA with vigorous agitation on a vortex mixer. The tissue was then passed through a 70 μm tissue strainer and the process repeated until the wash remained clear. The remaining lamina propria (LP) was digested in 1 mg/ml Collagenase Type VIII (Sigma Aldrich, C9722) for 20 min in an orbital shaker at 270 rpm and 37° C. Tissues were vortexed briefly and filtered to remove any remaining undigested material and cells counted prior to flow cytometric evaluation.

### Flow Cytometry

Cells from indicated compartments were incubated with fluorescent rat anti-mouse antibodies including against: mouse CD4 (RM4-5), CD8 (53-6.7), CD19 (1D3), Nk1.1 (PK136), CD11c (N418), MHCII (M5/114.15.2), F4/80 (BM8), Siglec F (E50-2440) and GR-1 (RB6-8C5) or their respective isotype controls. FoxP3 staining was performed according to manufacturer's instructions using the PE-FoxP3 labeling kit (eBiosciences, San Diego, Calif.). Cells were washed and fixed with 2% paraformaldehyde and analyzed using the FACS® Canto system (Beckton-Dickinson Immunocytometry Systems, San Jose, Calif.). Post-analyses were performed using FLOWJo software (Tree Star Inc, Ashland, Oreg.).

### Cytokine Production Assays

Tissue explants (0.5 cm2) were cultured for 24 h in DMEM (without sodium pyruvate, Cellgro Manassas, Va., supplemented with 5% FBS, 2 mM glutamine, 100 IU penicillin and 100 μg/ml streptomycin; Invitrogen) culture supernatants were then analyzed for the presence of cytokines using the Quansys Cytokine Assay system (Logan, Utah).

### Statistics

Statistical analyses were performed using Student t test or two-way ANOVA with Bonferroni posttests with Graphpad Prism Data Analysis software (GraphPad Software, La Jolla, Calif.). Data were expressed as mean±standard error of the mean (SEM). Statistical significance was set at a P value of less than 0.05.

All of the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method for treating a bowel disorder in a subject comprising, administering a therapeutically effective amount of alpha-1 antitrypsin (AAT) to the subject having a bowel disorder.

2. The method of claim 1, wherein the bowel disorder is an inflammatory bowel disease.

3. The method in claim 1, wherein the inflammatory bowel disease comprises Crohn's disease, ulcerative colitis (UC), collagenous colitis, lymphocytic colitis, ischaemic colitis, diversion colitis, behçet's syndrome, indeterminate colitis or a combination thereof.

4. A method for modulating weight loss in a subject having an inflammatory bowel disorder comprising:
identifying a subject having an inflammatory bowel disorder; and
administering a therapeutically effective amount of a composition comprising one or more of AAT, AAT mutant, or combination thereof, to the subject and modulating weight loss in the subject.

5. A method for modulating colon shortening in a subject having an inflammatory bowel disorder comprising:
identifying a subject having an inflammatory bowel disorder; and
administering a therapeutically effective amount of a composition comprising one or more of AAT, AAT mutant, or a combination thereof to the subject and modulating colon shortening in the subject.

6. The method of claim 5, wherein modulating colon shortening in the subject comprises reducing colon shortening in the subject compared to a control not receiving the composition.

7. A method for reducing cytokine production in a subject in need of such a treatment comprising:
identifying a subject having an IBD; and
administering a therapeutically effective amount of a composition comprising one or more of AAT, AAT-derived peptide, AAT mutant to the subject.

8. A method for preventing or reducing the risk of onset or progression of an IBD in a subject comprising, administering a therapeutically effective amount of a composition comprising one or more of AAT, AAT mutant, to the subject for preventing or reducing the risk of onset or progression of an IBD.

9. A method for treating a subject experiencing intestinal or bowel hyperpermeability comprising,
identifying a subject experiencing intestinal or bowel hyperpermeability due to a condition;
treating the subject with a therapeutically effective amount of a composition of alpha-1 antitrypsin (AAT) or carboxyterminal peptide thereof; and
restoring the intestinal or bowel function in the subject compared to a control subject not receiving the composition.

10. The method of claim 9, wherein the subject is treated with 10 mg/kg to 100 mg/kg of alpha-1 antitrypsin (AAT) or carboxyterminal peptide thereof at a predetermined interval.

11. The method of claim 9, wherein the condition is an acute condition.

12. The method of claim 9, wherein the condition is a chronic condition.

13. The method of claim 12, wherein the chronic condition comprises an inflammatory disorder.

14. The method of claim 12, wherein the chronic condition comprises inflammatory bowel disease.


SEQUENCE LISTING (31 sequences)

SEQ ID NO: 1 - protein, 394 aa, Homo sapiens
  misc_feature (356)..(357): Xaa can be any naturally occurring amino acid
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDHEEAKKQINDYVEKGTQGKIVDLVKELDRDTVFALVNYIFFKGKWERPFEVKDTEDEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLERXXRSIPPEVKFNKPFVFLMIEQNTKSPLFMGKVVNPTQK

SEQ ID NO: 2 - protein, 5 aa, Artificial
  synthetic peptide
FVFLM

SEQ ID NO: 3 - protein, 5 aa, Artificial
  synthetic peptide
FVFAM

SEQ ID NO: 4 - protein, 5 aa, Artificial
  synthetic peptide
FVALM

SEQ ID NO: 5 - protein, 5 aa, Artificial
  synthetic peptide
FVFLA

SEQ ID NO: 6 - protein, 5 aa, Artificial
  synthetic peptide
FLVFI

SEQ ID NO: 7 - protein, 5 aa, Artificial
  synthetic peptide
FLMII

SEQ ID NO: 8 - protein, 5 aa, Artificial
  synthetic peptide
FLFVL

SEQ ID NO: 9 - protein, 5 aa, Artificial
  synthetic peptide
FLFVV

SEQ ID NO: 10 - protein, 5 aa, Artificial
  synthetic peptide
FLFLI

SEQ ID NO: 11 - protein, 5 aa, Artificial
  synthetic peptide
FLFFI

SEQ ID NO: 12 - protein, 5 aa, Artificial
  synthetic peptide
FLMFI

SEQ ID NO: 13 - protein, 5 aa, Artificial
  synthetic peptide
FMLLI

SEQ ID NO: 14 - protein, 5 aa, Artificial
  synthetic peptide
FIIMI

SEQ ID NO: 15 - protein, 5 aa, Artificial
  synthetic peptide
FLFCI

SEQ ID NO: 16 - protein, 5 aa, Artificial
  synthetic peptide
FLFAV

SEQ ID NO: 17 - protein, 5 aa, Artificial
  synthetic peptide
FVYLI

SEQ ID NO: 18 - protein, 5 aa, Artificial
  synthetic peptide
FAFLM

SEQ ID NO: 19 - protein, 5 aa, Artificial
  synthetic peptide
AVFLM

SEQ ID NO: 20 - protein, 10 aa, Homo sapiens
GITKVFSNGA

SEQ ID NO: 21 - protein, 10 aa, Homo sapiens
DLSGVTEEAP

SEQ ID NO: 22 - protein, 10 aa, Homo sapiens
LKLSKAVHKA

SEQ ID NO: 23 - protein, 10 aa, Homo sapiens
VLTIDEKGTE

SEQ ID NO: 24 - protein, 10 aa, Homo sapiens
AAGAMFLEAI

SEQ ID NO: 25 - protein, 10 aa, Homo sapiens
PMSIPPEVKF

SEQ ID NO: 26 - protein, 10 aa, Homo sapiens
NKPFVFLMIE

SEQ ID NO: 27 - protein, 10 aa, Homo sapiens
QNTKSPLFMG

SEQ ID NO: 28 - protein, 8 aa, Homo sapiens
KVVNPTQK

SEQ ID NO: 29 - protein, 22 aa, Homo sapiens
LEAIPMSIPPEVKFNKPFVFLM

SEQ ID NO: 30 - protein, 20 aa, Homo sapiens
LEAIPMSIPPEVKFNKPFVF

SEQ ID NO: 31 - protein, 80 aa, Homo sapiens
GADLSGVTEEAPLKLSKAVHKAVLTIDEKGTEAAGAMFLERIPVSIPPEVKFNKPFVFLMIEQNTKSPLFMGKVVNPTQK